(19) United States
(12) Patent Application Publication
(10) Pub. No.: US 2008/0160096 A1
(43) Pub. Date: Jul. 3, 2008

(54) Polymeric nanoparticles by ion-ion interactions

(76) Inventors: Janos Berbely, Debrecen (HU); Magdolna Bodnar, Hajduboszormeny (HU); Istvan Hajdu, Tiszacsege, NJ (US); John F. Hartmann, Princeton Junction, NJ (US); Zsolt Keresztessy, Debrecen (HU); Laszlo Nagy, Debrecen (HU); Gyorgy Vamosi, Debrecen (HU)

Correspondence Address: Thomas A. O'Rourke;Bodner & O'Rourke, 425 Broadhollow Road, Melville, NY, 11747, US

(21) Appl. No.: 11/881,831

(22) Filed: Jul. 27, 2007

Related U.S. Application Data
(60) Provisional application No. 60/833,672, filed on Jul. 27, 2006.

(51) Int. Cl. A61K 9/50

(52) U.S. Cl. 424/493; 424/490; 514/44; 424/497; 435/440

(57) ABSTRACT

The present invention relates to biocompatible and biodegradable stimuli-sensitive polymeric nanoparticles, which were formed by ion-ion interaction in aqueous media. Synthetic and biological macromolecules with ionizable functional groups are capable of forming nanoparticles whose size and surface properties are sensitive to environmental influences such as pH, temperature and salt concentration. Nanodevices are designed for therapeutic applications as drug and nucleic acid carriers, and/or for MRI diagnosis as contrast agents. These nanodevices are designed for therapeutic applications as targeted drug carriers. Additionally, they can be used as contrast agents for MRI diagnosis. These nanosystems are also potential carriers for delivery of active ingredients as DNA, RNA, short interfering RNA (siRNA), antisense oligonucleotides (AS-ON), and triple helix forming oligonucleotides (TFO) etc. for pharmaceutical applications. Their adjustable size offers yet another advantage.

This application claims priority on U.S. Application Ser. No. 60/833,672 entitled “POLYMERIC NANOPARTICLES BY ION-ION INTERACTIONS” filed Jul. 27, 2006, the disclosures of which are incorporated herein by reference.

### FIELD OF INVENTION

The present invention relates to the preparation of nanoparticles from biopolymers such as polycations and polyanions via an ionotropic gellation process, for the purpose of encapsulating nucleic acid such as therapeutic DNA, RNA, siRNA, antisense oligonucleotides (AS-ON) etc., to achieve specific and intracellular delivery of such compounds and provide means of gene therapy.

### BACKGROUND OF THE INVENTION

There is an increasing demand for nanodevices, which are capable of carrying drugs and other therapeutic agents such as nucleic acids to tissue or cells. Recently many drugs have been discovered, which show good efficacy in treatment of cancer or other diseases, however, because of their serious side effects, healthy tissues and organs are affected. The targeted delivery of drugs and chemotherapies using nanodevices offers protection to healthy body segments and also allows dosage reduction. The present nanodevices with their sandwich-like structure are able to protect the active ingredient carried and their surface is designed to avoid immune reactions.

Gene therapy means the transferring of genetic material into specific cells of a patient to treat genetic diseases such as hemophilia, muscular dystrophy, cystic fibrosis, cardiovascular and neurological conditions, infectious diseases or cancer by replacing the errant genes, altering gene expression, producing cytotoxic proteins/pro-drug activating enzymes to stop cell proliferation, or vaccinating against viruses by the above means.

A critical barrier to clinical gene therapy is that an efficient and safe delivery vehicle remains to be discovered. Non-viral gene delivery vectors are superior to viral vectors (recombinant viruses) by providing improved safety, flexibility and facile manufacturing. Such polymer- or lipid-based vectors (usually polycations) electrostatically bind DNA/RNA and condense it into nano-sized particles (polyplexes or lipoplexes), protect the genes from degradation and mediate cellular entry.

Viral vectors allow a (i) high transfection rate and a (ii) rapid transcription of the foreign material inserted in the viral genome. However (i) safety issues have been raised following the death of a patient during a clinical trial, (ii) only small sequences of DNA can be inserted into the virus genome and (iii) large-scale production may be difficult. Finally, toxicity, immune and inflammatory responses can occur and insertional mutagenesis and oncogenic effects have also been observed in vivo.

Non-viral systems—synthetic and natural polycations offer (i) low immunogenicity, (ii) relatively large sequences may be condensed in small particles, (iii) good protection to DNA, (iv) easy manufacture, and may be modified to target specific cells and/or diseases. However several problems such as toxicity, lack of biodegradability, low yield of gene transfection, biocompatibility and in particular, low transfection efficiency need to be solved prior to practical use features in shuttling genes into cells.

Design criteria for non-viral vectors are as follows. Such systems have to protect nucleic acids from degradation, enable packaging of large DNA plasmids, provide for easy administration, support serum stability and targetability to specific cell types. Simplicity of fabrication and inexpensive synthesis and facile purification are also desired. They have to be robust/stable, facilitate internalization into cells, promote endolysosomal escape of the load, achive nuclear transport and efficient unpackaging for the function of the nucleic acids to be manifested. Infection of non-dividing cells is important in tissue therapy. Further important requirements include general safety, non-toxicity, non- or low immunogenicity and non-pathogenicity (Pack et al., 2005; Tiera et al., 2006; Barron-Peppas et al., 2007; Huang, 2005; Hetzline et al., 2004).

### SUMMARY OF INVENTION

There is an increasing demand for nanodevices, which are capable of targeting drugs to tissue or cells. Many drugs have been discovered, which show good efficacy in treatment of cancer or other diseases, however, because of their serious side effects, healthy tissues and organs are affected. The targeted delivery of drugs and chemotherapies using nanodevices offers protection to healthy body segments and also allows dosage reduction.

The present invention relates to the preparation of nanoparticles from biopolymers such as polycations and polyanions via an ionotropic gellation process, for the purpose of encapsulating nucleic acid such as therapeutic DNA, RNA, antisense oligonucleotides (AS-ON), small interfering RNA molecules (siRNA), triple helix forming oligonucleotides (TFO) etc., to achieve specific and intracellular delivery of such compounds to provide means of gene therapy. In the preferred embodiment, polycation (PC) is complexed with DNA and coated with polyanion (PA), via ion-ion interactions (FIG. 4). The present nanodevices with their sandwich-like structure are able to protect the active ingredient cargo. The polycation (PC) is complexed with DNA then coated with a polyanion (PA), via an ionotropic gellation process. The PA balances the surface charge, protects DNA, improves circulation time and shields the nucleic acid from the immune system. The self-assembling nature of the nanoparticle provides a simple means of (i) particle preparation without resorting to chemical cross-linking, organic solvents, or other toxic additives (ii) controlled degradation of the starting polymers facilitates particle size, and (iii) surface charge and functionality of the nanoparticles are conveniently controlled by varying mixing ratios of the components. The main components are natural and renewable polymers, which are biocompatible, biodegradable and likely non-immunogenic.

### BRIEF DESCRIPTION OF THE DRAWINGS

For a fuller understanding of the invention, reference should be made to the following detailed description, taken in connection with the accompanying drawings, in which:

FIG. 1 is a representation of nanoparticles formed by ion-ion interaction of polyelectrolyte macromolecules. A: positively charged polyelectrolyte (dark blue) on the surface. B: negatively charged polyelectrolyte (light blue) on the surface. The surface charge is determined by the sequence of mixing.

FIG. 2 is a schematic representation of nanodevice. The nanoparticle is loaded with DNA or RNA or siRNA, AS-ON, TFO etc. and targeting molecules.

FIG. 3 shows characterization of chitosan-FITC/poly-γ-glutamic acid-folate nanoparticles (CHIT: γ-PGA 1:1, 0.3 mg/ml). (a) AFM micrograph with color key for the third dimension, (b) TEM micrograph of particles and particle size distribution, and (c) the size distribution of the particles determined by DLS.

FIG. 4. depicts the preparation of nanodevice for DNA/RNA/siRNA delivery. Polycation (PC) is complexed with DNA then coated with a polyanion (PA), via an ionotropic gelation process. PC is labeled with fluorescein isothiocyanate (FITC) and single stranded DNA was labeled with Cy3 fluorescent dye for microscopic imaging. PA was conjugated with folic acid as cancer cell specific targeting moiety.

FIG. 5 is an atomic force microscopic image of CHIT-FITC/DNA-Cy3/PGA-FA nanoparticles.

FIG. 6 depicts nanoparticle assisted accumulation of the labeled oligonucleotide DNS-Cy3 in HeLa cells during 1, 3, 6 and 10 min. of incubation. Combined signals of visible, fluorescein (green) and Cy3 (red) from laser scanning confocal microscopy.

### DETAILED DESCRIPTION OF THE INVENTION

Macromolecules with ionizable functional groups such as carboxyl, amino, etc., in an aqueous medium form polycations and polyanions. Under specific conditions polycations and polyanions form nanoparticles by ion-ion interactions. The formation of nanoparticles requires specific reaction parameters, otherwise flocculation and precipitation occurs. However, once the nanoparticles were formed at specific pH and salt concentration, the nanosystem is stable.

### Sequence of Polyions

Ion-ion interaction can be performed between the functional groups of polyions, and the ratio of original polyions and the order of mixing can affect the particle structure and morphology. The linear polyelectrolyte chains can collapse in compact globules or can extend to coil conformations depending on the pH. The conformation of polymers is an important factor. The final formation of globular nanoparticles is dependent upon interactions between polyelectrolytes. Core-shell or sandwich like morphology can be obtained by varying the ratio of original polyions, the pH and the order of mixing.

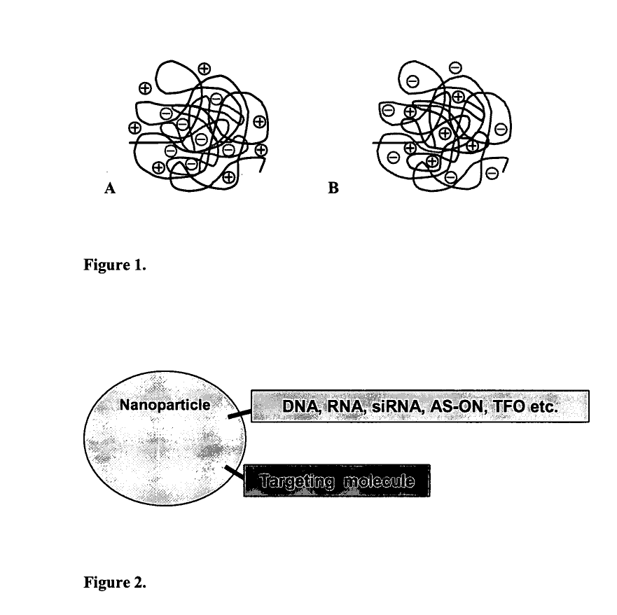

FIG. 1 shows a representation of nanoparticles formed by ion-ion interaction of polyelectrolyte macromolecules. A: positively charged polyelectrolyte (Dark blue lines) on the surface. B: negatively charged polyelectrolyte (Light blue lines) on the surface. The surface charge is determined by the sequence of mixing.

FIG. 2 is a schematic representation of nanodevice. The nanoparticle is loaded with DNA or RNA or siRNA, AS-ON, TFO etc. and targeting molecules.

### Adjusting of pH

The size of nanoparticles depends on the pH of the solution. The hydrodynamic diameter of nanoparticles increases by increasing the pH.

### Surface Charge

Surface charge of nanoparticles reveals the sequence of polyion addition. At lower pH, positively charged nanoparticles can be found, independently of the ratio of polyions or order of mixing. By increasing the pH, negatively charged nanoparticles are formed. The ratio of charged free functional groups determines the charge extent of nanoparticles, which depends on the pH and the ratio of functional groups.

### Salt Effect

The hydrodynamic diameter and the stability of nanoparticles were investigated in KCl solution. It was found that the hydrodynamic diameters decreased with increasing salt concentration, but the stability of the aqueous solutions was independent of the salt concentration.

### Adjusting the Concentration of Polyions

The stability of the aqueous solution and the size of nanoparticles depend on the original concentration of polyions. The hydrodynamic diameter of nanoparticles increases with increasing the original concentration of polyions. The stability of the aqueous solution decreases with increasing the original concentrations, and precipitation can be observed in some cases of mixing at high concentration of original polyions.

### EXAMPLE 1

### Nanoparticles Formed From Poly Acrylic Acid (PAA) and Polyammonium Salt (PAMM)

PAA with Mw=200 kDa and poly(2-methacryloxyethyltrimethylammonium bromide) were dissolved in water at a concentration of 1 mg/ml. The pH value of solutions was adjusted to pH 3 by 0.10 mol/dm³ sodium hydroxide. The solution of PAMM was added to the solution of PAA with gentle stirring. After 1 hour the pH was increased to 7 resulting in a stable nanosystem with particle size of 50 to 350 nm measured by laser light scattering method.

The size of nanoparticles is variable in a range of 10-1,000 nm by using polymers with different molecular weight. Also the particle size increased at higher pH due to the repulsion of negative charges.

### EXAMPLE 2

### Nanoparticles Formed From Chitosan (CHIT) and Poly gamma Glutamic Acid (PGA)

CHIT with Mv=320 kDa and PGA with Mw=1.3 MDa were dissolved in distilled water. The concentration was varied in the range 0.1 mg/ml-2.0 mg/ml. The pH value of the solutions was adjusted to pH=3 with 0.10 mol/dm³ hydrochloric acid. The ratio of polyelectrolyte and the order of mixing were modulated. After 1 hour mixing, the pH was increased with 0.1 M sodium hydroxide solution resulting in stable nanosystems. The hydrodynamic diameter of nanoparticles was in the range of 40-480 nm at pH 3, and at pH 7 was 470-1300 nm measured by laser light scattering method. There was some precipitation at higher pH caused by flocculation and coagulation.

The size of nanoparticles can be varied by using polymers with different molecular weights.

### EXAMPLE 3

### Nanoparticles Formed From CHIT and Hyaluronic Acid (HYAL)

CHIT with Mv=320 kDa and HYAL with Mw=2.5 MDa were dissolved in water. The concentration of CHIT was varied in the range 0.1 mg/ml-1.0 mg/ml, and of HYAL 0.04-0.2 mg/ml. The pH value of solutions was adjusted to pH 3 with 0.10 mol/dm³ hydrochloric acid. The ratio of polyelectrolyte and the order of mixing were modulated. After 1 hour mixing the pH was increased with 0.1 M sodium hydroxide solution resulting in stable nanosystems. The hydrodynamic diameter of nanoparticles was in the range of 130-350 nm at pH 3, and was higher than 600 nm at pH 7 as measured by laser light scattering. There was some precipitation at higher pH caused by flocculation and coagulation.

The size of nanoparticles can be varied by using polymers with different molecular weights.

### EXAMPLE 4

### Nanoparticles Formed From CHIT and Alginic Acid (ALGA)

CHIT with Mv=320 kDa and ALGA with Mv=30 kDa were dissolved in water. The concentration of CHIT was varied in the range 0.1 mg/ml-1 mg/ml, and of ALGA 0.04-0.2 mg/ml. The pH value of solutions was adjusted to pH=3 by 0.10 mol/dm³ hydrochloric acid. The ratio of polyelectrolyte and the order of mixing were modulated. After 1 hour mixing the pH was increased by 0.1 M sodium hydroxide solution resulting stable nanosystems at a pH=3. There was some precipitation at higher pH caused by flocculation and coagulation.

The size of nanoparticles can be varied by using polymers with different molecular weights.

### EXAMPLE 5

### Nanoparticles Formed From Modified CHIT and PGA

Chitosan was partially modified by betaine. The modification was performed by using the carbodiimide technique. CHIT was dissolved in hydrochloric acid media. Betaine was dissolved in water and then adjusted to pH 6.5 with 0.1 M sodium hydroxide solution. Water soluble carbodiimide was added to the betaine solution and the reaction was stirred for 30 min and subsequently mixed with the chitosan solution.

The modified CHIT and PGA with a Mw=1.3 MDa were dissolved in water. The concentration was varied in the range 0.1 mg/ml-2.0 mg/ml. The pH values of the solutions was adjusted to pH=3 with 0.10 mol/dm³ hydrochloric acid. The ratio of polyelectrolyte and the order of mixing were modulated. After 1 hour mixing the pH was increased by 0.1 M sodium hydroxide solution resulting in stable nanosystems. There was some precipitation at higher pH caused by flocculation and coagulation.

The size of nanoparticles can be varied by using polymers with different molecular weight.

### EXAMPLE 6

### Preparation of FITC-Labeled and Folic Acid (FA) Conjugated CHIT-PGA Nanoparticles

To assess the suitability of CHIT/PGA nanosystem for intracellular delivery of bioactive compounds including nucleic acids, first additional components were incorporated to allow e.g. cancer cell specific targeting and detection of cellular uptake. As a targeting moiety, the vitamin folic acid (FA) was chosen, which has a high affinity for folate receptors (FAR) which are overexpressed in a number of epithelial and myeloid cancer cells.

FA was conjugated to poly-γ-glutamic acid (MW 1.3 MDa, GPC) using water soluble carbodiimide. After the dropwise addition of EDC (1-Ethyl-3-[3-dimethylaminopropyl]carbodiimide hydrochloride, 8 mg in 1 ml dd. Water) to the γ-PGA solution (50 ml, 1 mg/ml, pH 6.5), the reaction mixture was stirred at room temperature for 30 min. FA (12 mg in DMSO) was added and stirred at room temperature for 24 h. The γ-PGA-FA conjugate was purified by dialysis and the number of FA molecules per γ-PGA was estimated by UV-VIS absorption spectroscopy (λₘₐₓ₁ 368 nm, ε9120; λₘₐₓ₂ 283 nm, ε25100). This showed that an average of 7 FA molecules was attached to one PGA molecules by this method.

Low molecular weight chitosan (MW 320,000 Da, as determined by viscosity measurements, and with a degree of deacetylation of 88%) solution (10 ml, 1 mg/ml in water, solubilised with HCl and pH adjusted to 6.5 with NaOH) was mixed with an aliquot of fluorescein isothiocyanate (FITC, 1 mg/ml in DMSO, 250 μl) and the reaction mixture was stirred at room temperature for 24 h. Fluorescein-labelled chitosan (CHIT-FITC) was purified by dialysis against water (MW cutoff 10 000 Da, 3 days) and characterised by UV-VIS spectrophotometry, which showed that 71 fluorescein molecules were attached per chitosan molecules by this method.

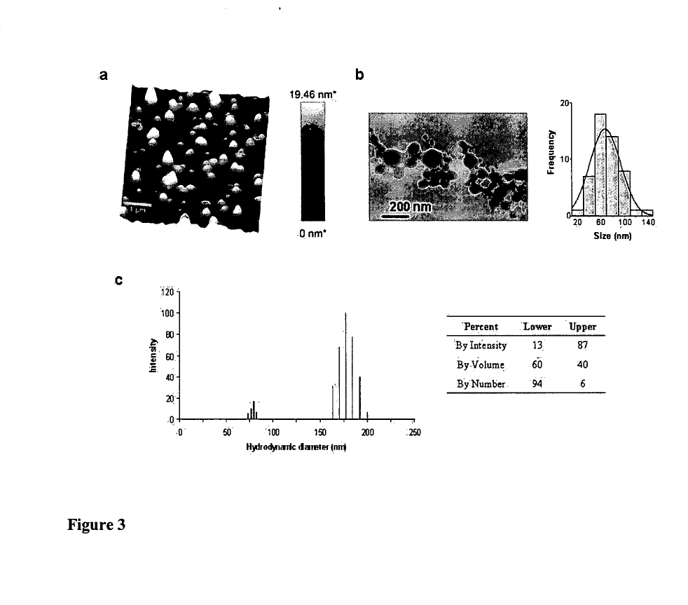

Stable self-assembled polyelectrolytes were developed via an ionotropic gelation process between the folated γ-PGA and the fluorescently labelled chitosan linear chains. When an equal volume of aqueous γ-PGA-FA (0.3 mg/ml, pH 9.0) and CHIT-FITC (0.3 mg/ml, pH 4.0) were mixed under continuous stirring, an opaque colloidal system was formed (75% transmittance at λ500 nm, pH 7.4), which remained stable at room temperature for several weeks at physiological pH. The presence of individual nanoparticles was confirmed and their size distribution characterized by transmission electron microscopy (TEM), atomic force microscopy (AFM) and dynamic light scattering (DLS) as described before. The analyses demonstrated that the CHIT-FITC/γ-PGA-FA nanosystem consists of spherical particles with a smooth surface both in aqueous environment and in a dried state (FIGS. 3a, b). TEM micrograph showed a size range of 30-160 nm with a mean value of 67.8 nm (FIG. 3b), while DLS reported a bimodal distribution for hydrodynamic diameter ranging between 70-90 nm and 160-200 nm with mean values of 80 and 178 nm, respectively (FIG. 3c). This is consistent with the particles swelling in an aqueous environment. The overall charge ratio of the nanoparticles (number of —NH₃⁺ groups of CHIT vs —COO⁻ groups on γ-PGA) was calculated as +0.67:−1 based on the weight ratio between CHIT and γ-PGA. This results in a negative zeta-potential at physiological pH, which may contribute to stabilisation of the nanosystem via charge repulsion between individual particles (Majoros et al., 2006; Hong et al., 2007; Hajdu et al., 2007; Berger et al., 2004; Hsieh et al., 2005; Lin et al., 2006; Lin et al., 2007).

FIG. 3 shows the characterisation of chitosan-FITC/poly-γ-glutamic acid-folate nanoparticles (CHIT:γ-PGA 1:1, 0.3 mg/ml). (a) AFM micrograph with color key for the third dimension, (b) TEM micrograph of particles and particle size distribution, and (c) the size distribution of the particles was determined by DLS.

### EXAMPLE 7

### Nanodevice for DNA Delivery

CHIT with Mv=320 kDa was dissolved in water at pH=3. An aqueous solution of DNA with Mw=32 kDa and with specific sequence was added. A stable nanosystem was formed. In the second step, PGA with Mw=1.2 MDa was added to cover the residual surface. The sandwich-like composite nanodevice containing the DNA molecules was stable at pH=7 and the NaCl concentration was 0.1 g/dm³.

### EXAMPLE 8

### Nanodevice for Single Stranded Oligonucleotides Delivery

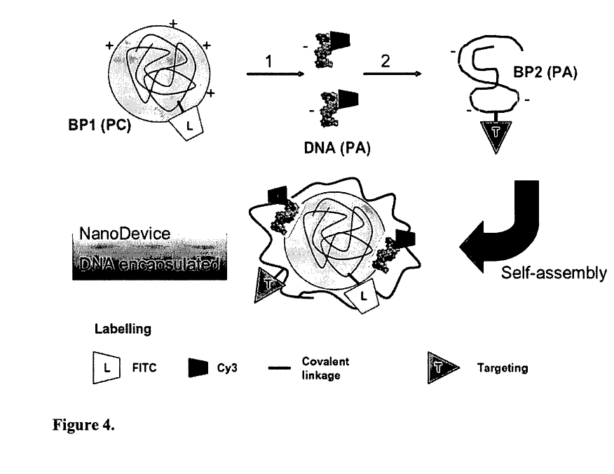

Nanoparticles were formed from CHIT, DNA and PGA by a general method represented in FIG. 4.

FIG. 4 shows the preparation of nanodevice for DNA/RNA/siRNA, antisense oligonucleoties etc delivery. Polycation (PC) is complexed with DNA then coated with a polyanion (PA), via an ionotropic gelation process. PC is labelled with fluorescein isothiocyanate (FITC) and single stranded DNA was labeled with Cy3 fluorescent dye for microscopic imaging. PA was conjugated with folic acid as cancer cell specific targeting moiety.

CHIT with Mv=320 kDa was labelled with FITC as described in Example 6. PGA with Mw=1.2 MDa was conjugated with folic acid (FA) as described in Example 6. Single stranded DNA consisting of 20 nucleotides and with a specific sequence was labelled at the 3′ end with Cy3 fluorescent dye (DNA-Cy3). CHIT-FITC was dissolved in water at a concentration of 0.3 mg/ml and at pH 4. DNA-Cy3 was dissolved in water at a concentration of 0.6 mg/ml and at pH 7.4. PGA-FA was dissolved in water at a concentration of 0.3 mg/ml and at pH 9.5. Nanoparticles were formed by either mixing 1 ml of CHIT-FITC solution pre-combined with 50 μl DNA-Cy3 solution and 1 ml of PGA-FA solution or by mixing 1 ml of CHIT-FITC solution and 1 ml of PGA-FA solution pre-combined with 50 μl DNA-Cy3 solution.

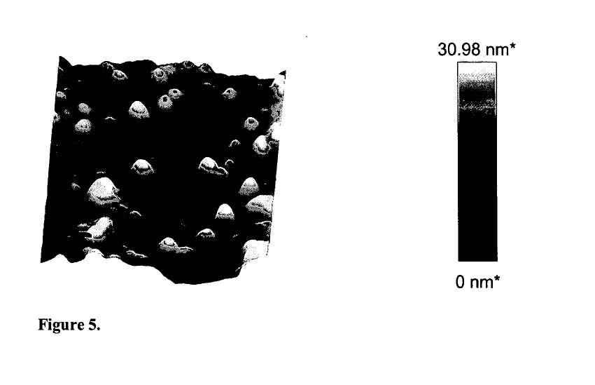

The nanosystems were characterised by dynamic light scattering (DLS) or atomic force microscopy (AFM). The results showed that both types of nanoparticles (which differ in the order in which components were mixed) had an effective diameter of 75-77 nm in aqueous environment (by DLS with a distribution between 46-148 nm) or up to 31 nm in a dried state (by AFM) as shown in FIG. 5. FIG. 5 shows an atomic force microscopic image of CHIT-FITC/DNA-Cy3/PGA-FA nanoparticles.

### EXAMPLE 9

### Intracellular Delivery of Single Stranded Oligonucleotides by CHIT/PGA Nanoparticles

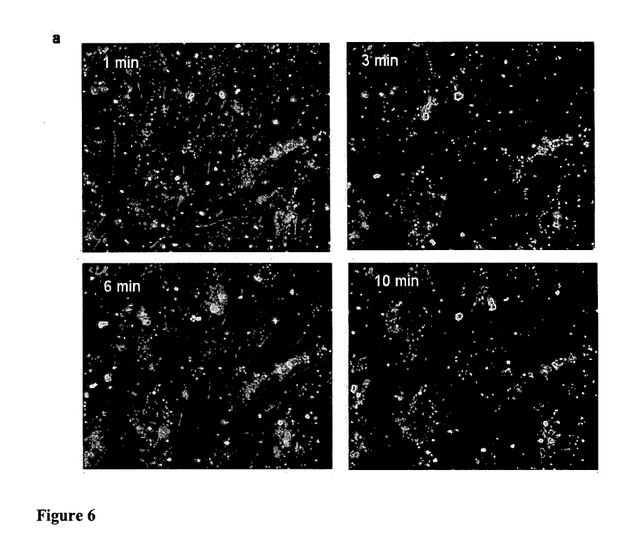

Nanoparticles from Example 8 were tested for intracellular delivery of single stranded oligonucleotides into the human cervical cancer cell line, HeLa, by laser scanning confocal microscopy (LSCM). Cells were cultured in RPMI 1640 medium (Sigma, St. Louis, Mo.) supplemented with 10% fetal bovine serum (Fisher Chemicals, Fairlawn, N.J.). Cells were grown at 37° C. in a humidified atmosphere of 5% CO₂ (v/v) in air. All experiments were performed on cells in the exponential growth phase. A culture of the cells were incubated with the nanoparticles for up to 30 min and the internalisation of the oligonucleotide (DNA-Cy3) as well as the CHIT-FITC was observed. The experiments clearly showed that the DNA was delivered into the cytoplasm of HeLa cells (FIG. 6). The form of entrance appeared to be vesicular giving rise to an uneven distribution of the DNA within the cytoplasm. After longer incubation, the DNA-Cy3 compound showed accumulation within the nucleus of the cells (FIG. 6 10 min). In control experiments, performed by exposing the cells to DNA-Cy3 without encapsulation within the nanoparticles showed that the DNA did not accumulate within the cells or nucleus.

FIG. 6 shows Nanoparticle assisted accumulation of the labelled oligonucleotide DNS-Cy3 in HeLa cells during 1, 3, 6 and 10 minutes of incubation. Combined signals of visible, fluorescein (green) and Cy3 (red) from laser scanning confocal microscopy.

Accordingly, a chitosan/poly-γ-glutamic acid-based self-assembling nanoparticulate system as a delivery platform for nucleic acids is provided. The two main components of this polycationic-polyanionic gel-type composite nanosystem are renewable as chitosan is derived from chitin of crustacean shell by alkaline deacetylation while γ-PGA is also easily obtained from Bacillus sp. ferments, where it is produced as slime. CS and γ-PGA are known to be fully biocompatible, biodegradable and likely non-immunogenic and also failed to display any toxicity in our cellular and in vivo studies. Also, the two polymers have a wide range of biomedical applications in separate or in combination. Additional advantages of the CS/γ-PGA nanosystem as nanocarrier are that (i) its self-assembling nature provides simple preparation without resorting to chemical cross-linking, organic solvents, or other toxic additives (ii) the use of degraded polymers facilitate particle size control, and that (iii) surface charge and functionality of NPs are conveniently tunable by varying component mixing ratios.

Thus, it will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

### REFERENCES

Barron-Peppas, L. et al. (2007) Encapsulation of nucleic acids and opportunities for cancer treatment. Pharmaceutical Res. 24, 618-627.
Pack, D. W. et al. (2005) Design and development of polymers for gene delivery. Nat. Rev. Drug Discovery 4, 581-593.
Tiera, M. J. et al. (2006) Synthetic and natural polycations for gene therapy: State of the art and new perspectives. Curr. Gene Therapy 6, 59-71.
Huang, M. (2005) Transfection efficiency of chitosan vectors: Effect of polymer molecular weight and degree of deacetylation. J. Controlled Release 106, 391-406.
Hetzline, S. E. et al. (2004) Progress in the development of nucleic acid therapeutics for cancer. Cancer Biol. Therapy 3, 4-12.
Majoros, I. J., Thomas, T., and Baker, J. R. Jr. (2006) Molecular engineering in nanotechnology: Engineered drug delivery. Handbook of Theoretical and Computational Nanotechnology, (Reith, M. and Schommers, W. Eds.) Vol 6, pp 673-717.
Hong, S., Leroueil, P. R., Majoros, I. J., Orr, B. G., Baker, J. R. Jr., and Banasak Holl, M. M. (2007) The binding avidity of a nanoparticle-based multivalent targeted drug delivery platform. Chem. Biol. 14, 107-115.
Hajdu, I., Bodnar, M., Filipcsei, G., Hartmann, J. F., Daroczi, L., Zrinyi, M., and Borbely, J. (2007) Nanoparticles prepared by self-assembly of chitosan and poly-γ-glutamic acid. Coll. Polym. Sci., in press.
Berger, J., Reist, M., Mayer, J. M., Felt, O., and Gurny, R. (2004) Structure and interactions in chitosan hydrogels formed by complexation or aggregation for biomedical applications. Eur. J. Pharm. Biopharm. 57, 35-52.
Hsieh, C.-Y., Tsai, S.-P., Wang, D.-M., Chang, Y.-N., Hsieh, H.-J. (2005) Preparation of γ-PGA/chitosan composite tissue engineering matrices. Biomaterials 26, 5617-5623.
Lin, Y.-H., Chung, C. K., Chen, C.-T., Liang, H.-F., Chen, S.-C., and Sung, H.-W. (2006) Preparation of nanoparticles composed of chitosan/poly-g-glutamic acid and evaluation of their permeability through caco-2 cells. Biomacromolecules 6, 1104-1112.
Lin, Y.-H., Mi, F.-L., Chen, C.-T., Chang, W.-C., Peng, S.-F., Liang, H.-F., and Sung, H.-W. (2007) Preparation and characterization of nanoparticles shelled with chitosan for oral insulin delivery. Biomacromolecules 8, 146-152.

We claim:

1. A method of preparing core-shell nanoparticles comprising complexing polycations with one or more nucleic acids and coating the complex with polyanion (PA), in an aqueous solution via ion-ion interactions.

2. The method according to claim 1 wherein the nucleic acid is a natural nucleic acid.

3. The method according to claim 1 wherein the nucleic acid is a synthetic nucleic acid.

4. The method according to claim 1 wherein the nucleic acid is a single stranded nucleic acid.

5. The method according to claim 1 wherein the nucleic acid is a double stranded nucleic acid.

6. The method according to claim 1 wherein the nucleic acid is DNA.

7. The method according to claim 1 wherein the nucleic acid is RNA

8. The method according to claim 1 wherein the nucleic acid is an antisense oligonucleotides (AS-ON).

9. The method according to claim 1 wherein the nucleic acid is a small interfering RNA molecule (siRNA).

10. The method according to claim 1 wherein the nucleic acid is a triple helix forming oligonucleotides (TFO).

11. The method according to claim 1 wherein said polycation is a polyammonium salt (PAMM).

12. The method according to claim 1 wherein said polycation is chitosan.

13. The method according to claim 12 wherein the weight ratio of chitosan to nucleic acid is about 10:1.

14. The method according to claim 13 wherein the chitosan component ranges in molecular weight from about 60 kDa to 320 kDa.

15. The method of claim 1, wherein said polyanion is selected from a group consisting of polyacrylic acid (PAA), poly γ glutamic acid (PGA), hyaluronic acid (HYAL) and alginic acid (ALGA).

16. The method according to claim 12 wherein said chitosan is reacted with betaine.

17. The method according to claim 12 wherein said chitosan is reacted with FITC.

18. The method according to claim 15 wherein said poly y glutamic acid is reacted with carbodiimide and folic acid.

19. The method according to claim 1 wherein said ion-ion interaction is by means of ionotropic gellation.

20. The method according to claim 1 wherein said polycation is chitosan and said polyanion is poly y glutamic acid (PGA)

21. The method according to claim 20 wherein the weight ratio of chitosan to poly γ glutamic acid is about 1:1.

22. The nucleic acid delivery system according to claim 21 wherein PGA component range in molecular weight about 80 kDa to about 1 300 kDa.

23. A method for performing gene therapy comprising encapsulating a nucleic acid in a nanoparticle formed from the reaction of a polycation (PC) and a polyanion (PA), via an ion-ion reaction

24. The method according to claim 23 wherein the polycation (PC) is complexed with DNA then coated with a polyanion (PA), via ionotropic gellation.

25. A nucleic acid delivery system comprising a nucleic acid encapsulated in a nanoparticle formed from the reaction of a polycation (PC) and a polyanion (PA), via an ion-ion reaction.

26. The system according to claim 25 wherein the polycation (PC) is complexed with DNA then coated with a polyanion (PA), via ionotropic gellation.

27. A method of delivering a nucleic acid to a cell comprising the steps of: providing a polycation; providing a nucleic acid; providing a polyanion; combining the polycation, the nucleic acid and the polyanion under conditions sufficient to form polycation-nucleic acid-polyanion complexes; and contacting a target cell with the polycation-nucleic acid-polyanion complexes.
